(19) United States
(12) Patent Application Publication
(10) Pub. No.: US 2001/0048926 A1
(43) Pub. Date: Dec. 6, 2001

(54) SYNTHETIC PEPTIDE VACCINES FOR DENTAL CARIES

(76) Inventors: DANIEL J. SMITH, NATICK, MA (US); MARTIN A. TAUBMAN, NEWTONVILLE, MA (US)

Correspondence Address: PATRICIA GRANAHAN, HAMILTON BROOK SMITH AND REYNOLDS, TWO MILITIA DRIVE, LEXINGTON, MA, 02173

(21) Appl. No.: 08/967,573

(22) Filed: Nov. 10, 1997

Related U.S. Application Data
(63) Continuation-in-part of application No. 08/057,162, filed on Apr. 30, 1993, now Pat. No. 5,686,075.
Application No. 08/057,162 is a continuation-in-part of application No. 07/877,295, filed on May 1, 1992, now abandoned.

(51) Int. Cl. A61K 39/00; A61K 39/38; A61K 39/02; A61K 39/385

(52) U.S. Cl. 424/190.1; 424/184.1

(57) ABSTRACT

Vaccine compositions and immunogenic compositions are described which are glucosyltransferase subunit vaccines for dental caries and which contain at least one peptide which corresponds to a sequence of glucosyltransferase containing aspartate 413, aspartate 415 or both aspartate 413 and aspartate 415. These subunit vaccines elicit antibodies which protect an immunized mammal from dental caries. Methods of provoking an immune response to intact glucosyltransferase are also described.

### GOVERNMENT FUNDING

Work described herein was supported by grant numbers DE-04733 and DE-06153 awarded by the National Institutes of Health, National Institute of Dental Research. The U.S. Government has certain rights in the invention.

### RELATED APPLICATIONS

This application is a continuation-in-part of U.S. application Ser. No. 08/057,162, filed Apr. 30, 1993, which is a continuation-in-part of U.S. application Ser. No. 07/877,295, filed May 1, 1992. The entire teachings of these priority applications are incorporated herein by reference.

### BACKGROUND OF THE INVENTION

Mutans streptococci have been convincingly implicated in the initiation of dental caries in humans. The ability of these organisms to accumulate and colonize on the tooth surface has been associated with the synthesis of glucans from sucrose. Glucans are synthesized by constitutively secreted glucosyltransferase (GTF) enzymes. These enzymes have been considered as potential components of a dental caries vaccine because of their role in the pathogenicity of Mutans streptococci. However, vaccines based on intact GTF have a variety of disadvantages such as the presence of inappropriate epitopes and excess molecular material that does not possess appropriate immunogenic features.

Although the exact basis for experimental protection with GTF-type vaccines is unclear, it is quite likely that protection involves functional inhibition of the catalytic and/or the glucan-binding activities of GTF. Epitopes associated with these functions would theoretically be primary targets for immunological attack, provided that the relevant sequences are located in molecular areas that are accessible to antibody. Subunit vaccines provide a method to block functional domains without inducing immunity to irrelevant or unwanted epitopes. It has been reported that synthetic peptide vaccines associated with catalytic or glucan-binding domains of GTF could protect rats from experimental dental caries (Taubman et al., Infect. Immun. 63:3088-3093 (1995)). One of the peptides that was successfully used as a vaccine (Smith et al., Infect. Immun. 62:5470-5476 (1994)) demonstrated a sequence containing an aspartic acid (aspartate 451 in S. mutans GTF-B) to which the glucosyl moiety of sucrose was covalently bound (Mooser et al., J. Biol. Chem. 266:8916-8922 (1991)).

Glucosyltransferases from mutans streptococci and dextransucrase from Leuconostoc mesenteroides bear similarity in sequence (Funane et al., Biochem. 32:13696-13702 (1993)). A catalytically active aspartate has been identified in L. mesenteroides dextransucrase by Funane and coworkers (Funane et al., Biochem. 32:13696-13702 (1993)) which was approximately 30-40 residues toward the N terminus of the site of the putative catalytic aspartate 451 described in mutans streptococcal GTF (Mooser et al., J. Biol. Chem. 266:8916-8922 (1991)). The comparable amino acid sequences in mutans streptococcal GTFs that surround the catalytically active L. mesenteroides aspartate are Asp 413 and/or 415 in S. mutans GTF-B (Funane et al., Biochem. 32:13696-13702 (1993)).

### SUMMARY OF THE INVENTION

This invention pertains to subunit vaccine compositions which elicit immune system responses in mammals to glucosyltransferase (GTF), an enzyme that is implicated in the formation of dental caries. Rather than using intact GTF as an immunizing agent, the vaccine composition or immunogenic composition is prepared from particular immunogenic portions (subunits) of GTF.

The invention relates to vaccine compositions and immunogenic compositions comprising at least one peptide consisting essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in a mammal to whom it is administered. In particular embodiments, the amino acid sequence is selected from the group consisting of GGYEFLLANDVDNSNPVVQ (SEQ ID NO: 1); ANDVDNSNPVVQAEQLNWL (SEQ ID NO: 2); and ANDVDNSNPVVQ (SEQ ID NO: 3). In a particularly preferred embodiment, 2 or more of the peptides are present and arranged on a core matrix of 3 or more lysines.

The invention also relates to vaccine compositions and immunogenic compositions comprising at least two peptides covalently attached to a peptidyl core matrix, wherein each peptide consists essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in a mammal to whom it is administered. In a particular embodiment, the amino acid sequence is selected from the group consisting of GGYEFLLANDVDNSNPVVQ (SEQ ID NO: 1); ANDVDNSNPVVQAEQLNWL (SEQ ID NO: 2); and ANDVDNSNPVVQ (SEQ ID NO: 3). In additional embodiments, the composition further comprises at least one additional immunologic component covalently attached to said peptidyl core matrix. For example, the additional immunologic component can be an immunogenic portion of a pathogen selected from the group consisting of diphtheria, pertussis, tetanus, measles and polio vaccines.

The invention also pertains to a method of provoking an immune response to glucosyltransferase in mammals comprising administering to a mammal at least one peptide consisting essentially of an amino acid sequence of glucosyltransferase comprising an amino acid selected from the group consisting of aspartate 413, aspartate 415, an equivalent of aspartate 413, an equivalent of aspartate 415, and combinations thereof, which is of sufficient length to raise an immune response in the mammal, thereby provoking said immune response. In a preferred embodiment, the immune response results in reduction of the colonization or accumulation of mutans streptococcal strains in the mammal to whom the peptide is administered.

The invention further pertains to a method of immunizing a mammal against dental caries comprising administering to the mammal at least one peptide consisting essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in the mammal.

### BRIEF DESCRIPTION OF THE FIGURES

FIG. 1 shows the ELISA reactivity of IgG antibody to the inducing peptide construct 21 days (one immunization) and 63 days (three immunizations) after the first injection of 43 day old rats. Individual rat antiserum designations (duplicate assays) are indicated on the abscissa. The peptide construct used to coat plates is indicated at the top of each frame. Reciprocal titers that were within the range of the sham sera (n=4) reactions with each antigen (<1:200) are indicated as baseline values.

FIG. 2 shows the ELISA reactivity of IgA antibody to the inducing peptide construct 21 days (one immunization) and 63 days (three immunizations) after the first injection. Individual rat saliva designations are indicated on the abscissa. The peptide construct used to coat plates is indicated at the top of each frame. Reciprocal titers that were within the range of the sham sera (n=4) reactions with each antigen (<1:4) are indicated as baseline values.

FIG. 3 is a graph showing anti-S. sobrinus GTF reactivity of sera from groups of rats injected with peptide constructs GGY (square), AND (triangle), CAT (inverted triangle), S. sobrinus GTF (circle), or sham-injected (diamond). Symbols represent the geometric mean IgG antibody levels of 63 day sera (n=4-5 per group), tested against S. sobrinus GTF at the dilutions indicated.

FIG. 4 shows cross-reactivity of antibody to GGY, AND and CAT peptide constructs. IgG antibodies induced by injection with GGY (diagonal cross hatching), AND (open box), SAND (intersecting cross hatching) or CAT (horizontal hatching) peptide constructs were reacted in ELISA with the peptide construct indicated at the top of each frame. Each bar indicates the mean of the reciprocal titer of 4-5 rat sera taken on day 63. Reciprocal titers <10² were within the error of sham sera (n=4) tested against the respective peptide.

FIG. 5 is a graph showing the percentage inhibition of the S. sobrinus GTF-mediated incorporation of ¹⁴C glucose from labelled sucrose into water-insoluble glucan by sera from peptide or GTF-injected rats. Sixty three day rat sera to GGY (n=4), AND (n=4), and CAT (n=2) peptide constructs were tested at 1:10 dilutions. A single 63 day serum from a rat injected with S. sobrinus GTF was tested at a dilution of 1:100. Data are expressed as the percentage ¹⁴C glucose incorporation of individual sera, compared with the mean ¹⁴C glucose incorporation by three 63 day sera from sham-injected rats. Asterisks indicate peptide-immunized groups that significantly differed from the sham immunized groups (p<0.02) in level of inhibition.

FIG. 6 is a graph showing the mean total and smooth surface caries scores of groups of rats (n=11-12/group) injected with S. sobrinus GTF, AND or GGY peptide constructs, or sham-injected, after 59 days of infection with S. mutans SJ32. The levels of statistical significance are indicated in parentheses for immunized groups, compared with sham-immunized groups.

FIG. 7 shows the amino acid sequences of L. mesenteroides dextransucrase and mutans streptococcal glucosyltransferases (GTF) in regions of aspartates associated with catalytic activity, as well as peptide (GGY, AND, SAND, CAT) sequences used in the present studies. Catalytic aspartates are indicated by underlining. Sequences have been adjusted to maximize homology.

FIG. 8 shows the results of epitope mapping of the GGY peptide. B cell epitopes are indicated by solid bars in the graph and by capital letters in the peptide title. Heptapeptides preincubated with anti-GGY serum are indicated on the Y axis.

### DETAILED DESCRIPTION OF THE INVENTION

The principal etiologic agents of the infectious disease, dental caries, are Mutans streptococci. These oral pathogens infect the oral cavity during early childhood and normally remain associated with the host's dentition for life. Mutans streptococci must colonize and then accumulate on the tooth surface in sufficient numbers to achieve dissolution of the enamel. After the initial colonization by Mutans streptococci on the tooth surface, the Mutans streptococci produce glucosyltransferase (GTF), an enzyme which catalyzes the synthesis of glucans from sucrose. In addition, S. mutans express cell surface proteins which serve as glucan binding sites. Glucans mediate much of the subsequent accumulation of Mutans streptococci on the tooth surface. This results in an increase in the numbers of potentially cariogenic bacteria in plaque. The metabolism of various saccharides by the accumulated bacterial mass results in excretion of significant amounts of lactic acid as a metabolic product, which causes demineralization when present in sufficient amount in close proximity to the tooth surface. This eventually results in a carious lesion (a cavity).

The compositions of the present invention, e.g., vaccine compositions and immunogenic compositions, comprise at least one peptide consisting essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in a mammal to whom it is administered. The desired effect of these compositions is interruption of the cariogenic process, resulting in reduction, i.e., lessening or prevention, of dental caries.

Glucosyltransferases of the Mutans streptococci are particularly well-suited for application of synthetic vaccine technology for dental caries. The primary sequences of several mutans streptococcal GTFs have been deduced from DNA studies (Ferretti et al., Infect. Imm., 56:1585-1588 (1988); Russell et al., J. Dental Res., 67:543-547 (1988); Uoda et al., Gene, 69:1101-1109 (1988)). Although GTFs are large molecules, they function through a few discrete sites, which include the catalytic and glucan-binding sites. Primary sequences have been identified which provisionally include these sites (Mooser et al., J. Dental Res., 69:325 (1990); Russell et al., J. Dental Res., 67:543-547 (1988)).

As used herein, a vaccine composition is a composition which elicits an immune response in a mammal to which it is administered and which protects the immunized mammal against subsequent challenge by the immunizing agent or an immunologically cross-reactive agent. Protection can be complete or partial (i.e., a reduction in symptoms or infection as compared with an unvaccinated mammal). An immunologically cross-reactive agent can be, for example, the whole protein (e.g., glucosyltransferase) from which a subunit peptide used as the immunogen is derived. Alternatively, an immunologically cross-reactive agent can be a different protein which is recognized in whole or in part by the antibodies elicited by the immunizing agent.

As used herein, an immunogenic composition is intended to encompass a composition which elicits an immune response in a mammal to which it is administered and which may or may not protect the immunized mammal against subsequent challenge with the immunizing agent.

Peptides which are particularly useful in the present invention are peptides which consist essentially of an amino acid sequence of GTF comprising an amino acid selected from the group consisting of aspartate 413, aspartate 415, equivalents of aspartate 413, equivalents of aspartate 415and combinations thereof. For example, the amino acid sequence can be the amino acid sequence of the GGY peptide (GGYEFLLANDVDNSNPVVQ; (SEQ ID NO: 1)), the AND peptide (ANDVDNSNPVVQAEQLNWL; (SEQ ID NO: 2)), or the SAND peptide (ANDVDNSNPVVQ; (SEQ ID NO: 3)). Appropriate amino acid sequences will contain one or more of aspartate 413, aspartate 415 or equivalents of aspartates 413 or 415. As examples, appropriate amino acid sequences can contain aspartate 413, aspartate 415, an equivalent of aspartate 413, an equivalent of aspartate 415, aspartate 413 and aspartate 415, an equivalent of aspartate 413 and an equivalent of aspartate 415, aspartate 413 and an equivalent of aspartate 415 or aspartate 415 and an equivalent of aspartate 413.

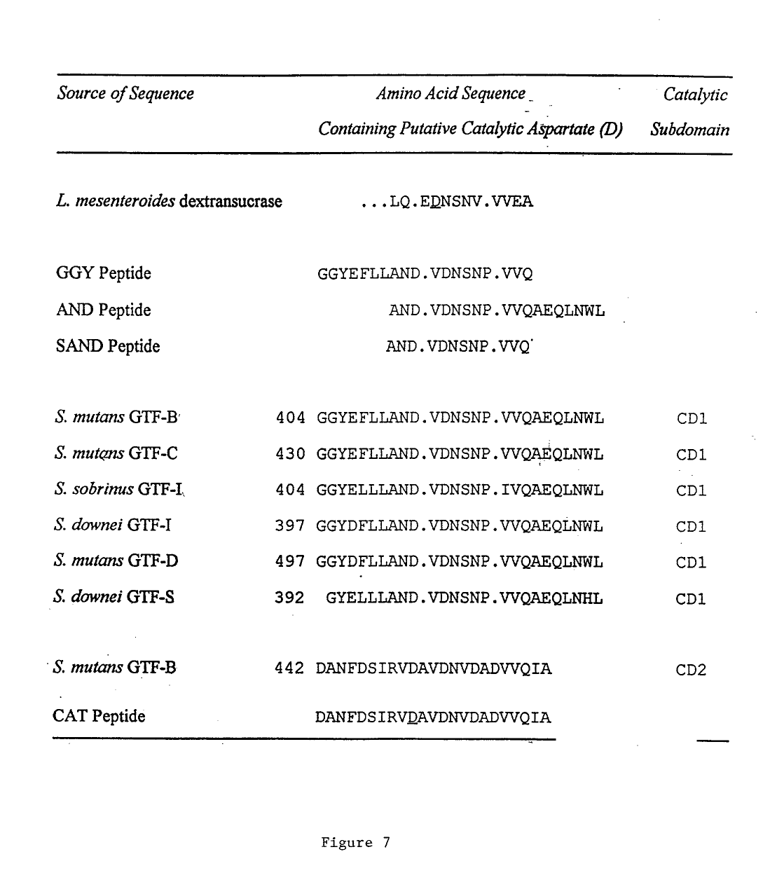

Aspartate 413 and aspartate 415 refer to the aspartate residues at amino acid positions 413 and 415, respectively, of S. mutans GTF-B. As used herein, equivalents of these aspartate residues are intended to include catalytic aspartate residues present at equivalent sites (positions) in other mutans streptococcal GTFs. That is, the amino acid position numbers of the aspartate residues can be different from 413 and 415 in other mutans streptococcal GTFs. These equivalent aspartate residues can be identified, for example, by aligning the amino acid sequences of other streptococcal GTFs (see, for example, FIG. 7) based on homology to S. mutans GTF-B. In addition, the characterization of the catalytic properties of an aspartate which is equivalent to aspartate 413 or 415 can be determined by methods described herein or methods known in the art (see, for example, Funane et al., Biochem. 32:13696-13702 (1993)).

Useful peptides will be of sufficient length to raise an immune response in a mammal to whom it is administered but will be less than the complete amino acid sequence of the intact GTF enzyme. Typically, the peptide will be at least 5-7 amino acids in length. Preferably the peptide will be at least 12 amino acids in length; more preferably the peptide will be at least 19 amino acids in length.

The immune response which is raised can comprise a B cell response, a T cell response or both a B cell and T cell response. The B cell response is associated with the appearance of mucosal antibody which is predominately IgA and systemic antibody which is predominantly IgG. The antibodies elicited by immunization will preferably recognize both the immunizing agent and an immunologically cross-reactive agent. In a preferred embodiment the antibody response will be sufficient to protect the immunized mammal against subsequent challenge or infection with the immunizing agent or an immunologically crossreactive agent.

Although the vaccine composition of the present invention can contain one peptide consisting essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in a mammal to whom it is administered, preferred embodiments of the vaccine composition of the present invention contain two or more of such peptides.

Those skilled in the art will be able to determine other immunologic domains of GTF, as well as additional immunologic components of non-GTF origin which enhance adjuvanticity or produce an immunogenic response against other infectious agents, suitable for use in the vaccine composition. For example, the peptides disclosed herein can be valuably combined in a vaccine or immunogenic composition with a CAT or GLU peptide or surface binding domain peptide such as those disclosed in U.S. Pat. No. ______ (Attorney Docket No. FDC92-01A). In particular embodiments, the vaccine or immunogenic composition of the present invention, can comprise an additional immunologic component which is an immunogenic portion of a pathogen such as, but not limited to, diphtheria, pertussis, tetanus, measles and polio virus, resulting in a multivalent vaccine producing protection against greater than one infectious disease or agent. Ultimately, a multivalent vaccine can be produced which incorporates relevant protective epitopes and appropriate adjuvant sequences targeting early childhood infections.

The peptides present in the vaccine composition of the present invention may be designed in a number of ways to enhance immunogenicity. In one embodiment in which the vaccine composition contains one or more peptides, the peptide is conjugated to a known protein, (such as tetanus toxoid) or a carrier (such as a synthetic polymer carrier) to give a macromolecular structure to the vaccine which thereby enhances immunogenicity. In a preferred embodiment in which the vaccine composition contains at least two peptides, the peptides are synthesized and covalently attached to a peptidyl core matrix to yield a macromolecule with a high density of peptides in a single structure. Each peptide in such a structure consists essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in a mammal to whom it is administered. The peptidyl core matrix can consist of amino acids such as lysine, arginine and histidine. In particular, at least 2 peptides are synthesized on a core matrix of at least one lysine to yield a macromolecular vaccine composition. Particularly, at least 2 peptides are synthesized on a core matrix of 3 lysines. In a preferred embodiment, a vaccine composition is designed in which four peptides of the present invention are synthesized and covalently attached to a core matrix of 3 lysines yielding a radially branched peptide with four dendritic arms. In this embodiment, the four peptides present can be the same or different. Those skilled in the art will be able to determine other variations of synthesizing and covalently attaching vaccine compositions of the present invention to a peptidyl core matrix by employing routine experimentation.

The present invention also pertains to pharmaceutical compositions comprising at least one peptide consisting essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in a mammal to whom it is administered. For instance, the peptide of the present invention can be formulated with a physiologically acceptable medium to prepare a pharmaceutical composition. The particular physiological medium may include, but is not limited to, water, buffered saline, polyols (e.g., glycerol, propylene glycol, liquid polyethylene glycol) and dextrose solutions. The optimum concentration of the active ingredient(s) in the chosen medium can be determined empirically, according to procedures well known to medicinal chemists, and will depend on the ultimate pharmaceutical formulation desired. Methods of introduction of exogenous peptides at the site of treatment include, but are not limited to, intradermal, intramuscular, intraperitoneal, intravenous, subcutaneous, oral and intranasal. Other suitable methods of introduction can also include rechargeable or biodegradable devices and slow release polymeric devices. The pharmaceutical compositions of this invention can also be administered as part of a combinatorial therapy with other agents.

The present invention further relates to a method of provoking an immune response to glucosyltransferase in a mammal by administering a peptide consisting essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in a mammal to whom it is administered. Preferably, the immune response results in interference with the enzymatic activity of glucosyltransferase in mammals after administration of the vaccine composition. The immune response elicited by the method of the present invention results in reduction of the colonization or accumulation of mutans streptococcal strains in the mammal to whom the vaccine or immunogenic composition is administered.

The invention also relates to a method of immunizing a mammal against dental caries comprising administering a peptide consisting essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in the mammal, to the mammal.

The compositions of the present invention can be administered to any mammal in which the prevention and/or reduction of dental caries is desired. Suitable mammals include primates, humans, cats, dogs and other mammals in whom it is desirable to inhibit dental caries. The present invention provides a vaccine that is useful for preventing, halting or reducing the progression of dental caries in a mammal to whom the vaccine is administered.

In the method of the present invention of provoking an immune response to glucosyltransferase, mammals in which an immune response to glucosyltransferase is desired are given the vaccine or immunogenic compositions described herein. The vaccine composition can be included in a formulation which is administered to an individual being treated; such a formulation can also include a physiologically compatible carrier (e.g., a physiological buffer), stabilizers, flavorants, adjuvants and other components. The vaccine can be administered by a variety of routes (e.g., parenterally, intravenously, orally) and the components of the formulation will be selected accordingly. The amount to be administered and the frequency of administration can be determined empirically and will take into consideration the age and size of the mammal being treated and the stage of the dental caries disease (e.g., prior to colonization of Mutans streptococci, soon after colonization of Mutans streptococci or in later stages of colonization).

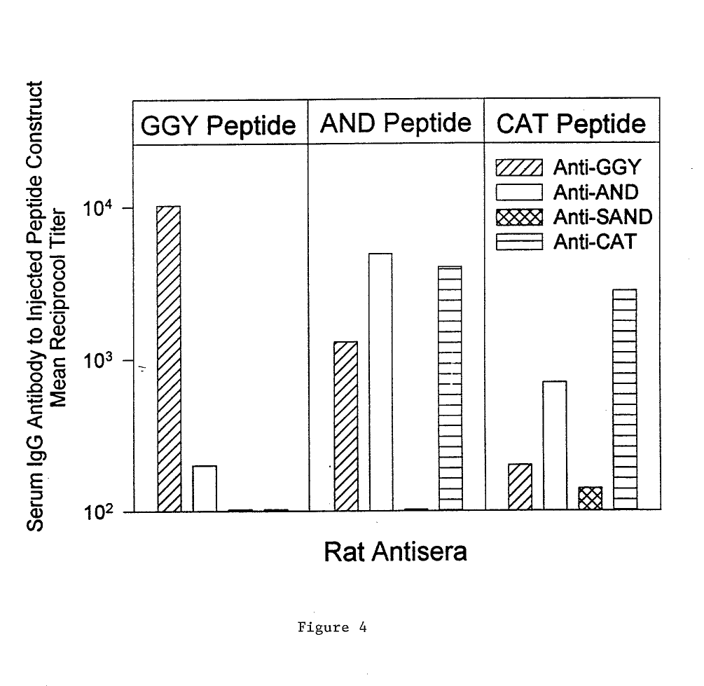

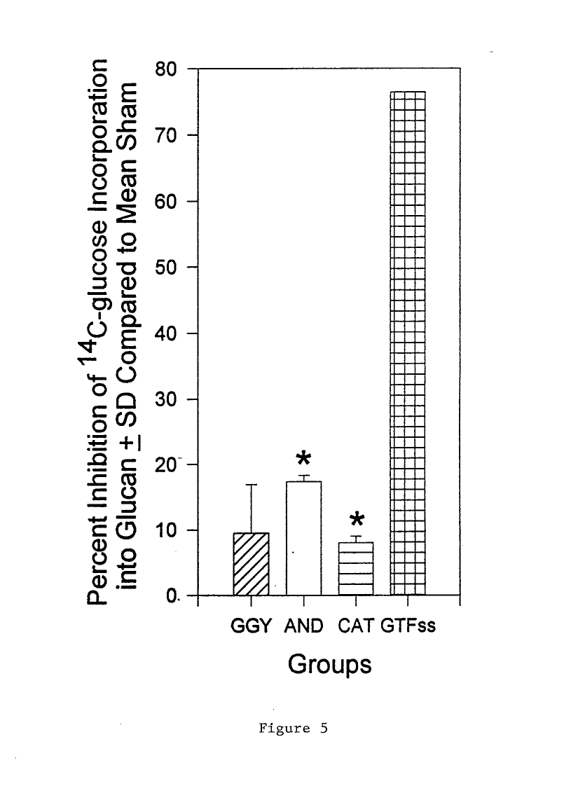

The results described herein support the hypothesis that Asp 413 and/or Asp 415 participate in the catalytic activity of mutans streptococcal GTF. Antibody raised to GGY or AND not only reacts with, but also inhibits the enzymatic activity of, GTF (FIG. 5). Furthermore, the results described herein demonstrate that antibodies to AND/GGY react with epitopes within the CAT peptide (FIG. 4). Furthermore, work herein demonstrates inhibition of insoluble glucan formation by GTF from S. sobrinus by polyclonal antibody (FIG. 5), strongly suggesting a catalytic role for one or both aspartates in the mutans streptococcal GTF sequence defined by GGY/AND/SAND.

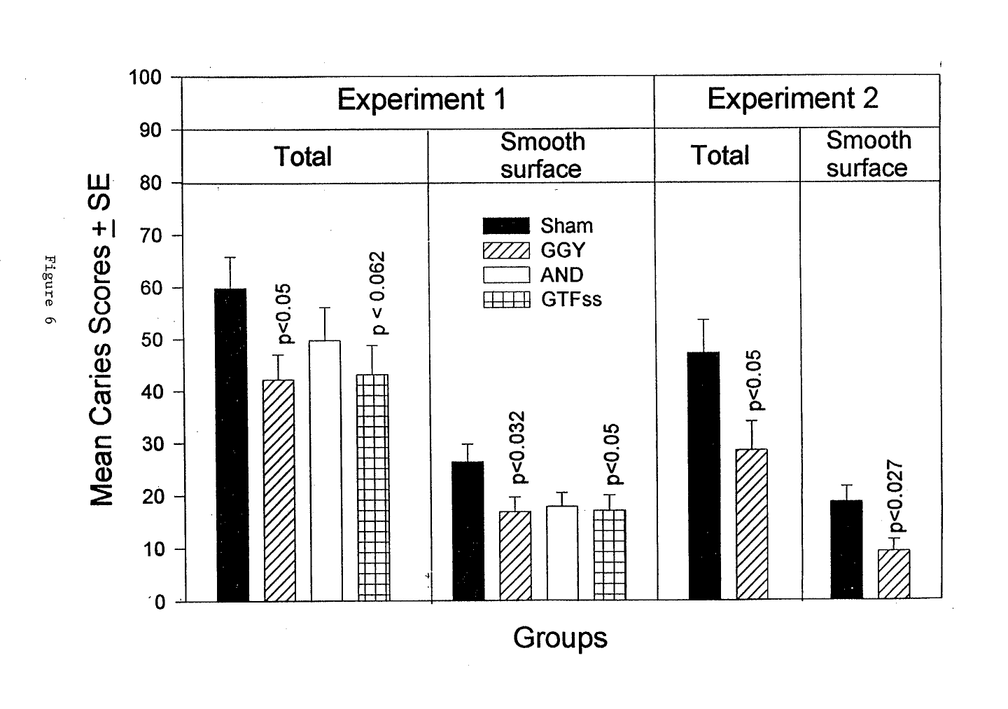

Epitopes expressed by the putative GGY/AND catalytic subdomain also induced protective immunity after infection of rats with cariogenic S. mutans (FIG. 7). Protection was at a level similar in magnitude to that achieved by injection with the intact GTF from S. sobrinus, illustrating the effectiveness of polyclonal antibody raised to an important functional determinant. The GGY peptide construct induced a response that gave significant protection, presumably because this peptide was more efficient than the AND construct in the induction of antibody formation (FIG. 6). The extent of protection afforded by GGY immunization was similar to that previously observed by immunization with CAT in a similarly executed protocol (Taubman, M. A. et al. Infect. Immun., 63: 3088-3093 (1995)), further emphasizing the immunogenicity, and perhaps, functional significance of these two sites.

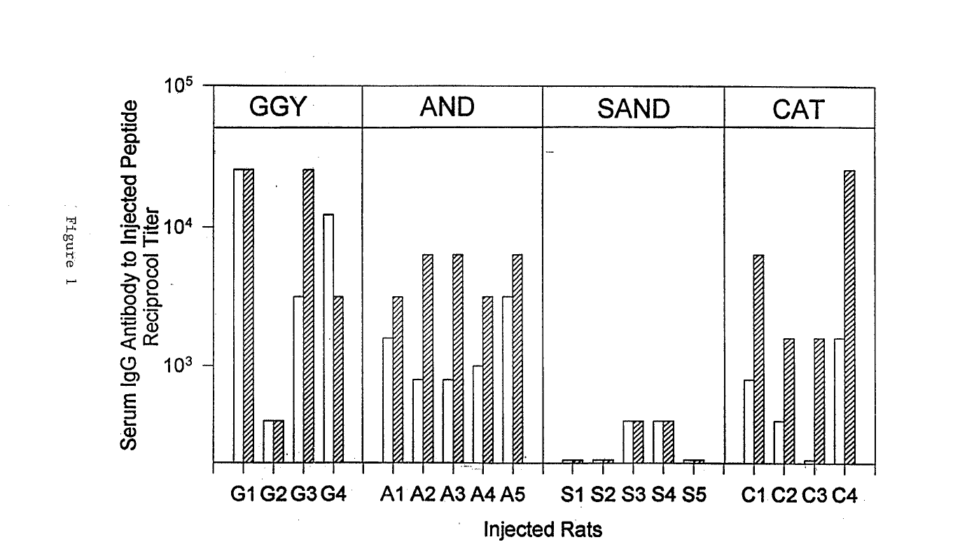

The age of the rat during which the peptide immunization protocol was applied and/or the time during which the rats was allowed to respond, affected the antibody levels observed. Weanling rats given two GGY or AND injections eight days apart and bled 14 days (day 39 of life) after the onset of immunization (protection experiment 1) demonstrated modest levels of serum IgG antibody to GGY and little detectable antibody to the AND construct. In contrast, weanling rats given two GGY injections 14 days apart and bled 26 days (day 51 of life) after the onset of immunization (protection experiment 2) had significantly elevated levels of serum IgG antibody (8/11 positively responding rats). Responses in this group of rats were similar to those observed in the sera of rats (4/4 responding rats) whose immunization protocol began on day 43 and were bled 21 days later (day 64 of life; FIG. 1, immunogenicity experiment). Despite these differences GGY immunization protocols of both protection experiments 1 and 2 resulted in significant protection from S. mutans-induced dental caries. Previously, using a protocol similar to experiment 1 to immunize rats with the CAT peptide, a significant protective effect was shown (Taubman, M. A. et al. Infect. Immun., 63: 3088-3093 (1995)), despite relatively low levels of serum IgG or salivary IgA antibody. It is likely that humoral antibody levels were significantly higher at infectious challenge, which began on day 53, of life since considerable antibody to GGY was detected in experiment 2 at this time. Alternatively, it is possible that the protocol used for protection experiment 1 served chiefly to prime rats and that subsequent stimulation by oral infection or residual antigen in the adjuvant upregulated responses that contributed to the protection seen.

Preliminary studies (Taubman et al., J. Dent. Res. 76:347 (1997)) indicate that multiepitopic peptide constructs, consisting of both CAT and GLU peptide branches induce enhanced immune responses. This strategy also could be used to increase the immune potential of the AND/GGY sequence described in the present study. Moreover, the combination of sequences from several strains into a synthetic or recombinant multi-epitopic construct could increase the protective potential of subunit vaccines for dental caries.

The present invention is illustrated by the following Examples, which are not intended to be limiting in any way. The teachings of all references cited herein are incorporated by reference in their entirety.

### EXAMPLES

Peptide Constructs: Four peptides were synthesized. GGY and AND were 19-mer peptides whose sequences overlapped an equivalent aspartate of L. mesenteroides dextransucrase which was shown to be catalytically active (Funane et al., Biochem. 32:13696-13702 (1993); FIG. 7). SAND was a 12-mer with sequence shared by GGY and AND (FIG. 7). CAT was a 21-mer peptide (Smith et al., Infect. Immunity 62:5470-5476 (1994)) containing the catalytically important Asp 451 (Mooser et al., J. Biol. Chem. 266:8916-8922 (1991)) which was completely homologous with the derived sequence of residues 444-464 of S. downei GTF-I (Ferretti et al., J. Bacteriol. 169:4271-4278 (1987)), and residues 442-462 of S. mutans GTF-B (Shiroza et al., J. Bacteriol. 169:4263 4270 (1987)). Peptides were synthesized (Applied Diagnostics, Foster City, Calif.) using the stepwise solid phase method of Merrifield (Merrifield, J. Amer. Chem. Soc. 85:2149-2154 (1963)) on a core matrix of lysines to yield macro molecules with four (CAT) or eight (GGY, AND, SAND) identical peptides per molecule, after the method of Tam (Tam, Proc. Natl. Acad. Sci. USA 85: 5409-5413 (1988)). Purity (>90%) was assessed using HPLC, amino acid analysis, and molecular weight determination by mass spectrometry.

Glucosyltransferases: Glucosyltransferases (GTF) from S. sobrinus strain 6715 were purified from culture supernatants after bacterial growth in glucose-containing defined media, as described in Taubman et al. (J. Oral Pathol. 17:466-470 (1988)). Enzymes were separated from other culture supernatant proteins by affinity chromatography on Sephadex G-100 (Pharmacia Fine Chemicals), using 6M guanidine HCl as the eluting solvent. GTFs were then obtained from the guanidine HCl eluate by FPLC chromatography on Superose 6 (Pharmacia) using 6M guanidine for elution. GTF preparations contained a mixture of GTF-I, GTF-Sd, and GTF-Si.

Immunogenicity of Peptides: Sprague Dawley CD strain 43 day-old male rats (Charles River Laboratories) were used for injection. Six groups of 4-5 rats were injected subcutaneously in the vicinity of the salivary glands with 50 μg each of GGY, AND, SAND or CAT peptide constructs, 10 μg of GTF, or sham-immunized with buffer alone. The initial injection included complete Freund adjuvant (CFA; Difco); two subsequent injections at 21-day intervals included incomplete Freund adjuvant. Animals were bled prior to injection and 14-21 days after each injection. Serum from coagulated and centrifuged blood was stored frozen at −20° C. until use.

ELISA: Serum IgG and salivary IgA antibodies were tested by enzyme-linked immunosorbent assay (ELISA). Polystyrene microtiter plates (Flow Laboratories) were coated with 2.5 g/ml of each peptide construct or 0.5 μg of S. sobrinus GTF. Antibody activity was then measured by incubation with 1:100 to 1:10⁶ dilutions of sera, or 1:4 to 1:32 dilutions of saliva. Plates were then developed for IgG antibody rabbit anti-rat IgG followed in sequence by alkaline phosphatase goat anti-rabbit IgG (Biosource Inc.) and p-nitrophenylphosphate (Sigma). A mouse monoclonal reagent to rat α chain (Zymed, South San Francisco, Calif.) was used with biotinylated goat anti-mouse IgG (TAGO, Inc., Burlingame, Calif.) and avidin-alkaline phosphatase (Cappel) to reveal levels of salivary IgA antibody to peptides. Reactivity was recorded as absorbance (A_{405 nm}) in a micro plate reader (Biotek Instruments). Data are reported as absorbance (A_{405 nm}), reciprocal titer, or as ELISA units (EU) which were calculated relative to the levels of appropriate reference sera or salivas from Sprague Dawley rats thrice immunized with the respective peptide construct. Dilutions of 1:12,800 and 1:6,400 (A_{405 nm} approximately 1.0) were considered 100 EU for serum IgG to the GGY and AND constructs, respectively. Dilutions of 1:32 were considered 100 EU for salivary IgA to both GGY and AND constructs.

Antibody Inhibition of Glucan Synthesis: Selected rat sera were evaluated for their ability to inhibit glucan synthesis catalyzed by S. sobrinus GTF, using a filter assay. Ten μl volumes of sera (1:100 dilutions in 0.02M sodium phosphate buffered saline and 0.02% sodium azide [PBSA], pH 6.5) were preincubated with the respective GTF for 1 hour at 37° C. in a total volume of 0.04 ml PBSA. Then 1.7 mg sucrose, 44 nCi of [¹⁴C-glucose]-sucrose (approximately 100,000 cpm) were added in 0.2 ml PESA in the presence of 37 μg of dextran T10 (Pharmacia). Incubation proceeded for 18 hours at 37° C., after which water-insoluble glucan was collected on Whatman GF/F glass fiber filters, washed with PBSA, and radioactivity determined as described in Smith et al. (Infect. Immunity 62:5470-5476 (1994)).

Epitope Mapping: B cell epitopes were mapped on the GGY and AND peptides by an ELISA inhibition technique. Anti-GGY or anti-AND sera were preincubated with 13 separate heptamers representing the incremental peptide sequence of the respective peptide, or with intact peptide, followed by the conventional ELISA with intact peptide-coated plates. B cell epitopes were indicated by a significant reduction in absorbance.

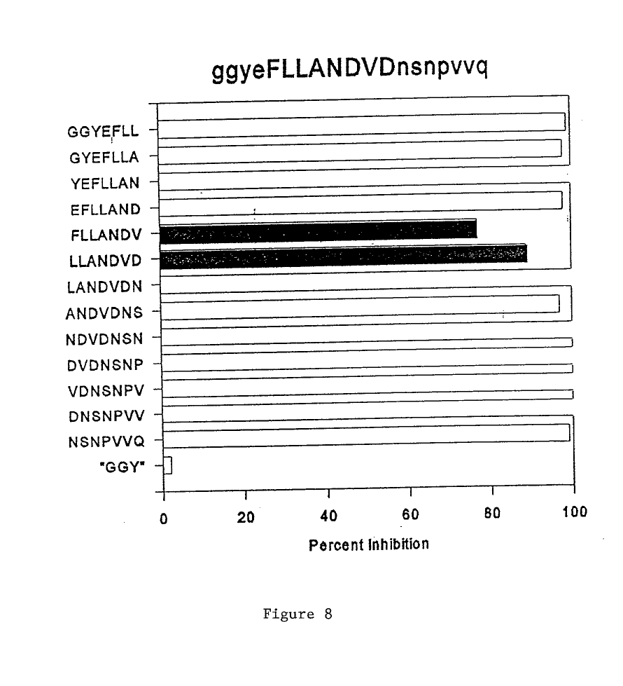

Heptapeptides FLLANDV and LLANDVD significantly inhibited the GGY antibody, indicating the presence of an epitope in the portion of the peptide containing the putative catalytic residues (FIG. 8). The reaction could be completely inhibited with intact GGY. Mapping the AND construct revealed an N terminal epitope that partly overlapped the GGY epitope.

Experimental Protocol for Protection Experiments: Animals used in the present experiments were derived from germ-free Sprague-Dawley rats that had been reared in the isolator facility of Charles River Laboratories, Wilmington, Mass. (Area 051). A population of these rats were found to be free of indigenous mutans streptococci. These rats served as initial breeding stock for dams used in these experiments and were regularly monitored for absence of mutans streptococci. The progeny of the dams used for the present experiments were weaned at approximately 21 days and were subsequently fed high sucrose Diet 2000.

Two experiments were performed. Groups (n=11-12) of 23-26 day-old rats were subcutaneously (sc) injected in the salivary gland vicinity with (experiment 1) GGY (50 μg), AND (50 μg) or GTF(10 μg), or phosphate buffered saline (control animals), or (experiment 2) GGY (50 ;μg) or PBS. Each antigen was incorporated with complete Freund adjuvant in both experiments. Eight (experiment 1) or 14 (experiment 2) days later, rats were reinjected with PBS or with the same antigen and dose in incomplete Freund adjuvant. Six (experiment 1) or 12 (experiment 2) days after the second injection, animals were bled from the tail vein, and saliva was collected after injection of pilocarpine (1.0 mg/100 g body weight). Fourteen (experiment 1) or 11 (experiment 2) days after the second injection, all rats were orally infected with approximately 10⁸ S. mutans for 3 consecutive days. Both experiments were terminated 63 days after initial infection.

Bacterial recoveries: The mutans streptococcal flora was assessed at termination as previously described (Taubman et al., Infect. Immun. 63:3088-3093 (1995)). After systematic swabbing of teeth, sonication, and plating appropriate dilutions on mitis salivarius agar (MS; total streptococci), and MS agar with 0.006 mg/ml bacitracin (MSB; mutans streptococci), plates were incubated for 48 hours at 37° C. in 90% N₂, 10% C0₂. Mutans streptococcal CFUs were then enumerated microscopically on MSB agar.

Caries assessment: The extent and depth of carious lesions in all rat molar teeth (caries score) were microscopically evaluated by a modified Keyes method as previously described (Taubman et al., J. Immunol. 118:710-720 (1977)). Caries scores were determined separately on smooth and on sulcal dental surfaces.

FIG. 1 presents the IgG titers of all peptide-injected rat sera when assayed against the homologous peptide construct after one (day 21, open bar) and three (day 63: hatched bar) subcutaneous injections of peptide. Immunization was begun when rats were 43 days of age. Both eight-branched 19-mer GGY and AND constructs induced serum IgG antibody to the inciting antigen after the first injection in all rats. Subsequent immunization increased serum IgG titers in all AND-injected rats. Interestingly, a single immunization with GGY was sufficient to induce maximal serum IgG antibody to GGY under the conditions of the assay. Immunogenicities, both in terms of percentage of animals responding and titer of responding sera, of GGY and AND peptides were at least as high as observed for the positive control CAT peptide whose sequence is based on the CD2 subdomain (FIG. 7). The 12-mer SAND peptide appeared to be weakly immunogenic (FIG. 1).

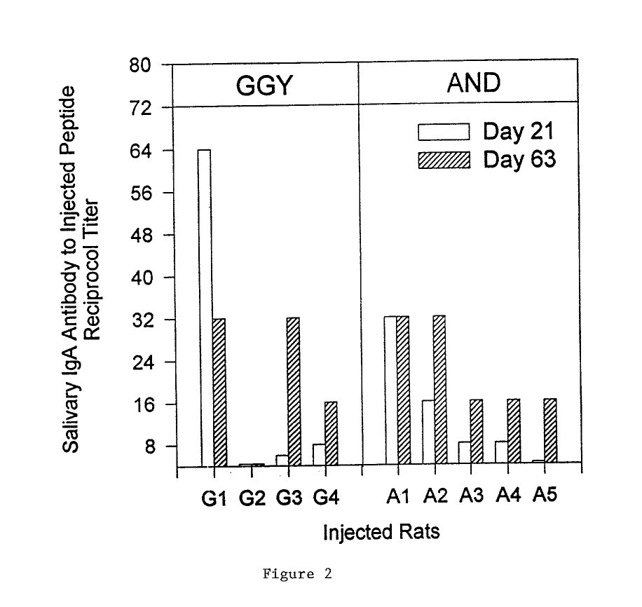

FIG. 2 presents the IgA titers of GGY and AND-peptide construct-injected rat saliva, assayed the homologous peptide construct after one (day 21) and three (day 63) subcutaneous infections. Significant levels of salivary IgA antibody could be detected in 7/9 rats by day 21 and 8/9 rats by day 63. Again, a single immunization was sufficient to induce maximal titers in one rat of both groups. Thus, GGY and AND peptide eight-branched constructs induced significant levels of serum IgG and salivary IgA antibody to the inciting antigen.

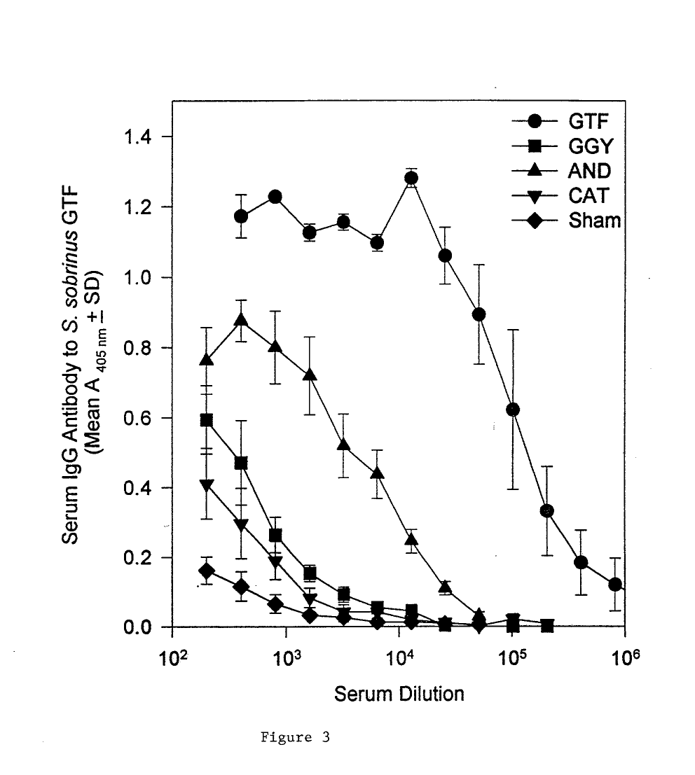

All antisera to immunogenic peptides were also assayed in ELISA for IgG antibody that would react with intact S. sobrinus GTF (FIG. 3). Sera from groups of rats injected with GGY, AND or CAT peptides each demonstrated significant reactivity with GTF. Antisera to the AND peptide were significantly more reactive with GTF than serum antibody induced by the GGY or CAT peptides. GTF-reactive IgG antibody levels to the latter two peptides were similar to one another. These observations indicate that the GGY and AND sequences from the CD1 subdomain present epitopes that are similar to those of intact GTF.

The antigenic similarity of epitopes in the CD1 and CD2 subdomains was explored by reacting antisera to GGY, AND, or SAND, which are derived from sequences containing Asp413 and Asp415, with ELISA plates coated with these peptide constructs or with the CAT peptide construct whose sequence includes the catalytic aspartate (Asp451 ) (FIG. 4). Serum IgG antibody from rats immunized with 19-mer peptides from the CD1 subdomain (GGY, AND) showed low but significant reaction with the CAT peptide. Conversely, serum IgG antibody from CAT.(CD2) and GGY (CD1) each showed significant reaction with the AND peptide construct (CD1). Antisera showed the least crossreactivity with the GGY peptide construct (CD1), although reaction with antisera induced by AND was observed. These data suggest that epitopes associated with CD2 (CAT) bear some antigenic similarity with epitopes associated with CD1 (GGY,AND).

Sera from rats injected with GGY, AND, or CAT peptides were also evaluated for their ability to inhibit the formation of water-insoluble glucan from sucrose by S. sobrinus GTF (FIG. 5). Sera from all rats injected with AND peptide inhibited GTF activity (p<0.01). Inhibition with sera from rats injected with GGY peptide was more variable, although one rat serum gave considerable (35%) inhibition of GTF activity. The level of inhibitory activity of AND-injected rats exceeded that observed with sera from rats similarly immunized with CAT (p<0.02), although inhibitory levels of sera from all peptide-injected rats were far below those induced by the intact GTF protein.

Two experiments were performed to measure the effect of immunization with the eight-branched GGY and AND peptide constructs on experimental dental caries in the Sprague-Dawley rat. In the first experiment, groups of rats were immunized with GGY or AND peptide constructs. In the second experiment groups of rats were immunized only with the GGY peptide construct. Pre-infection sera and salivas were collected for antibody measurement in 39 day old rats in experiment 1 (6 days after the second injection) and 51 day old rats in experiment 2 (12 days after the second injection). In the GGY-immunized rat groups, IgG antibody levels to GGY (experiment 1=21.9+17.8 EU; experiment 2=108.0±4.0 EU) were elevated above those of the sham-immunized groups (experiment 1=1.7±0.2 EU; experiment 2=4.0±3.0 EU) in both experiments and reached statistical significance (p<0.002) in experiment 2. Salivary IgA antibody levels to GGY in the GGY-injected groups (experiment 1=5.0±2.0 EU; experiment 2=11.7±4.8 EU) were also elevated above those of the sham-immunized groups (experiment 1=2.0±1.0 EU; experiment 2=2.0±1.0 EU), although differences did not reach statistical significance. Serum IgG or salivary IgA antibody levels to the AND construct were not significantly elevated.

Levels of S. mutans colonization were evaluated by swabbing rats 59 days after initial infection. The median infection levels in the GGY and AND-injected groups ranged between 33-48% of the median S. mutans infection level of the sham group. However, the differences did not reach significance because of the broad range of values.

FIG. 6 presents the total molar and smooth surface caries scores of all groups in experiments 1 and 2. The mean and median total, smooth surface, and occlusal caries scores of both AND- or GGY-injected groups were lower than the respective scores of the sham-injected group in both experiments. Total molar caries scores of the groups injected with the GGY construct were significantly lower (p<0.05) than those of the sham-injected groups in both experiments (FIG. 6). Protection was most evident on smooth surfaces of GGY-injected groups (experiment 1: p<0.032; experiment 2:p<0.027). Significant smooth surface caries reductions (p<0.045) were also observed in the group injected with S. sobrinus experiment GTF 1 (experiment 1). Thus, injection with peptides sequences containing the putative catalytic Asp 413 and Asp 415 can induce protective immunity for dental caries.

Equivalents

Those skilled in the art will recognize or be able to ascertain using no more than routine experimentation, many equivalents to the specific embodiments of the invention described herein. Such equivalents are intended to be encompassed in the scope of the following claims.

We claim:

1. A vaccine composition comprising at least one peptide consisting essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in a mammal to whom it is administered.

2. A vaccine composition according to claim 1 wherein the amino acid sequence is selected from the group consisting of:
a) GGYEFLLANDVDNSNPVVQ (SEQ ID NO: 1);
b) ANDVDNSNPVVQAEQLNWL (SEQ ID NO: 2); and
c) ANDVDNSNPVVQ (SEQ ID NO: 3).

3. A vaccine composition according to claim 1 wherein 2 or more of the peptides are present and arranged on a core matrix of 3 or more lysines.

4. A vaccine composition according to claim 1 wherein the immune response produces antibodies of the IgG or the IgA isotype.

5. A vaccine composition comprising at least two peptides covalently attached to a peptidyl core matrix, wherein each peptide consists essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in a mammal to whom it is administered.

6. A vaccine according to claim 5 wherein the amino acid sequence is selected from the group consisting of:
a) GGYEFLLANDVDNSNPVVQ (SEQ ID NO: 1);
b) ANDVDNSNPVVQAEQLNWL (SEQ ID NO: 2); and
c) ANDVDNSNPVVQ (SEQ ID NO: 3).

7. A vaccine composition according to claim 5 further comprising at least one additional immunologic component covalently attached to said peptidyl core matrix.

8. A vaccine composition according to claim 5 wherein the additional immunologic component is an immunogenic portion of a pathogen selected from the group consisting of diphtheria, pertussis, tetanus, measles and poliovirus.

9. A vaccine composition according to claim 5 wherein the additional immunologic component is selected from the group consisting of the CAT peptide, the GLU peptide and both the CAT and GLU peptide.

10. A vaccine composition according to claim 5 wherein the peptidyl core matrix comprises at least one lysine.

11. A vaccine composition according to claim 1 wherein the immune system response to glucosyltransferase results in the reduction of the colonization or accumulation of mutans streptococcal strains in a mammal to whom the vaccine composition is administered.

12. A vaccine composition according to claim 5 wherein the immune system response to glucosyltransferase results in the reduction of the colonization or accumulation of mutans streptococcal strains in a mammal to whom the vaccine composition is administered.

13. A method of provoking an immune response to glucosyltransferase in mammals comprising administering a peptide consisting essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in the mammal, to the mammal, which thereby provokes said immune response.

14. A method according to claim 13 wherein said immune response results in reduction of the colonization or accumulation of mutans streptococcal strains in the mammal to whom the peptide is administered.

15. A method of immunizing a mammal against dental caries comprising administering a peptide consisting essentially of an amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in the mammal, to the mammal.

16. An immunogenic composition comprising a peptide consisting essentially of at least one amino acid sequence of glucosyltransferase comprising an amino acid selected from the group consisting of aspartate 413, aspartate 415, an equivalent of aspartate 413, an equivalent of aspartate 415, and combinations thereof, and which is of sufficient length to raise an immune response in a mammal to whom it is administered.

17. An immunogenic composition according to claim 16 wherein the amino acid sequence is selected from the group consisting of:
a) GGYEFLLANDVDNSNPVVQ (SEQ ID NO: 1);
b) ANDVDNSNPVVQAEQLNWL (SEQ ID NO: 2); and
c) ANDVDNSNPVVQ (SEQ ID NO: 3).

18. An immunogenic composition according to claim 16 wherein 2 or more of the peptides are present and arranged on a core matrix of 3 or more lysines.

19. An immunogenic composition according to claim 16 wherein the additional immunologic component is selected from the group consisting of the CAT peptide, the GLU peptide and both the CAT and GLU peptide.
